(19) United States
(12) Patent Application Publication
(10) Pub. No.: US 2002/0124299 A1
(43) Pub. Date: Sep. 12, 2002

(54) Connection system for diver's mask

(76) Inventor: John Joseph Malcolm, Redwood City, CA (US)

Correspondence Address: John Joseph Malcolm, 1704 Carleton Court, Redwood City, CA, 94061-2666, US

(21) Appl. No.: 10/043,940

(22) Filed: Jan. 11, 2002

Related U.S. Application Data
(60) Provisional application No. 60/261,152, filed on Jan. 11, 2001.

(51) Int. Cl. A61F 9/02

(52) U.S. Cl. 2/439

(57) ABSTRACT

A secure fastener for use with divers' hoods comprises a post assembly (3000) and a latch assembly (2700). The post assembly comprises an inner post (2555), an outer post (3015), and a locking pin (2900). The inner post is placed inside the diver's hood (125). A hollow column (2567) on the inner post passes through a hole in the hood material. The outer post is joined with the inner post via the column. The column contains recesses (2565) that mate with an inner ridge (2550) on the outer post (3015). It also contains cuts (2570) that relieve the column walls and allow the inner and outer posts to slidably engage. The inner and outer posts are forced together, engaging the ridge with the lowest recess on the column. A locking pin (2900) is then inserted into the column, preventing flexure of the column walls and locking the two halves of the post assembly in place. The post assembly is thus able to accommodate different wall thicknesses encountered in diving hoods. Projections (2560), present on the inner surfaces of the inner and outer posts, prevent rotation of the assembly. A latch assembly (2700) is slidably engaged to the upper flange (2530) on the outer post. The latch can rotate on the post without disengaging The latch can be disengaged only by deliberately lifting a finger (2710) thereon. Thus the latch and post assembly are secure, rotatable, and can be removed only by deliberate action of the diver.

### CROSS-REFERENCE TO RELATED APPLICATIONS

This application claims the benefit of the filing date of Provisional Patent Application Ser. No. 60/261,152, Filed Jan. 11, 2001.

### FEDERALLY SPONSORED RESEARCH

None

### SEQUENCE LISTING OR PROGRAM

None

### BACKGROUND OF THE INVENTION

### Field Of Invention

This invention relates generally to diving masks, and in particular to a method and apparatus for firmly securing a mask to a diver's hood while permitting the mask to be moved from the diver's eye area without being detached and lost.

### BACKGROUND—Prior Art—Diving Mask Requirements

Divers frequently wear wet suits or dry suits and other paraphernalia, such as a tank, buoyancy compensating device, breathing regulator, hood, fins, and gloves. Just prior to or once in the water, the diver puts on his or her fins. One of the last pieces of diving gear that a diver puts on before entering the water is the mask. Fins are put on just prior to, or once in the water because it is very difficult to walk with them on.

After all the diving gear is on, the diver must hold and carry the mask and two fins until ready to enter the water. Diving gloves make it difficult to hold items and work with one's hands. The diver is likely to drop the mask or fins before entering the water. Also, any last-minute adjustments to the diver's gear necessitate putting the fins or mask down. If the mask is dropped or put down on a dirty surface, it may need to be rinsed off before it can be worn. This is very awkward when the diver is wearing gloves and other diving gear. If the diver stumbles or falls, she or he may not be able to break the fall because his or her hands are full.

The diver does not put on the mask until ready to enter the water because it is not possible to breathe through the nose with the mask on. Also any breath exhaled through the nose will fog the inside of the mask—requiring the diver to remove and defog it. Further, wearing the mask greatly diminishes the diver's upward, downward, and peripheral vision, resulting in a safety hazard before entering the water.

If the diver places the mask on top of his or her head for temporary storage, it is likely to fall off because there is no secure way to attach the mask to the hood. If the diver places the mask and strap around her or his neck it is very uncomfortable, and it is very difficult to put the mask on one's face from this location

If the diver is wearing a neoprene hood, it is difficult to properly place a silicone mask strap around the rear portion of the head because it tends to stick to the hood, and can become twisted. This problem is difficult to see and correct, especially if the diver is wearing gloves.

When a diver is under water, another diver can knock the mask off the diver's face by collision with an object, by swimming through and getting caught on kelp, and so forth. If the mask is knocked off the diver's head, visibility is greatly reduced and the mask can be lost. Even if the wearer or another diver finds the mask, it can be difficult to reinstall properly because of sticking between the strap and the hood. Again, this difficulty is compounded if the diver is wearing gloves.

While at the surface, it may be necessary for the diver to remove the mask. This is done in order to improve vision, to breathe through the nose, to defog the mask, to remove water that has leaked past the seal formed between the mask and the diver's face, or to reposition a mask that will not stay in place.

Placing the mask on top of the diver's head creates a risk of having it knocked off by a wave, or pushed off by the water while the diver swims. This is complicated by the fact that if the diver is wearing a hood, they will not be able to sense that the mask is being removed from their head. Placing the mask under the diver's chin is uncomfortable and restricts the ability to move the divers head, and it is difficult to reinstall the mask from this position.

Holding the mask with one hand restricts the diver's activity. The mask can slip out of the diver's hand without the diver realizing it, especially if they are wearing gloves.

### Background—Prior—Art—Goggle Mounting System

In U.S. Pat. No. 4,686,712 (1987) Spiva shows a goggle mounting system for a cyclist's helmet. The goggles are attached to the helmet by a pair of straps, one on each side. One strap is attached to the helmet by a keyhole and post (K&P) fastener. The other strap is attached to the helmet by a hook-and-loop (H&L) fastener. Separating only the H&L fastener permits the user to remove the goggles from their eyes, while keeping the goggles attached to the helmet by the keyhole and post fastener. In this condition, the goggles hang downward from one side of the helmet.

While this arrangement is suitable for cyclists, it is not useful for divers. In preparation for entering the water, it is preferred for divers to place their mask on top of their head. When they are ready to enter the water, the divers move the mask over their eyes. While the K&P fastener can swivel, the H&L fastener cannot. In order to move the mask to the top of the diver's head, the H&L fastener must be disengaged, the mask position changed, then the fastener re-engaged. This awkward maneuver is made more cumbersome when the diver wears gloves, as is frequently the case. In addition, when the H&L fastener is disengaged, the K&P fastener is loose and can also disengage. This can cause the diver to drop the mask, or lose it altogether at the surface or under water. Because the H&L is on the side of the diver's head, it is not possible for the diver to visually align or determine whether the fastener is fully engaged. Also, if one of the two straps were to break the mask could be easily lost because the remaining H&L or K&P could disengage.

### Background—Prior—Art—Fastener

In U.S. Pat. No. 2,311,231 (1943), Illsche shows a fastener for garments and other articles of flexible material. Illsche employs a bottom plate, a top plate, and a post. The bottom plate is placed on one side of the article, and the top plate is placed on the opposite side of the article. Each plate has a hole with coarse threads. The two plates are aligned and a post with threads or thread-like projections is screwed axially through both, joining them.

While it would be possible to place such a fastener on a diver's hood, and secure a mask to the hood using straps and K&P fasteners in conjunction with the posts, such an arrangement would not be suitable for use by divers. The act of moving the mask from the diver's face to the top of her or his head would tend to unscrew the post, causing loss of the mask. In addition, as above, the K&P fastener is unsuitable because it is easily disengaged and can cause loss of a mask when the diver is hit by a wave, jumps into the water, is kicked by another diver, swims through and gets entangled in kelp, and the like. Illsche's device was designed for articles of relatively constant thickness. Additionally, the K&P design leaves all parts exposed so the assembly can get entangled in kelp, fishing line, and the like while diving.

### Background—Objects and Advantages

Accordingly, one object and advantage of the present invention is to provide an improved method and apparatus for securing a diver's mask to a hood. Another object is to provide a mask and attachment, which is less susceptible to loss. Further objects are to provide a locking fastener which can be adjusted to fit articles of various thicknesses, which swivels without releasing, securely holding the mask while permitting it to be moved from the diver's face to the top of his or her head, which is easily engaged and released, to provide a fastener which can be engaged and released by a diver who is wearing gloves, and which will not disengage in response to trauma such as occurs when the diver's head is kicked by another diver, the diver swims through and gets entangled in kelp, or the diver simply jumps into the water. A further object is to provide two locking fasteners, attached to straps on either side of a mask, the other end of the straps being connected to the mask with strap tension adjusted at the mask end of the straps.

Additional objects and advantages will become apparent from a consideration of the drawings and ensuing description

### SUMMARY

In accordance with the invention, a diver's mask uses a post and latch (P&L) fastener to secure a mask to a diver's hood. The hood material can have a range of thicknesses. Two posts are attached to the diver's hood on opposite sides of the diver's head. Latches, which mate to the posts, are attached to straps, which in turn are attached to the diver's mask, with tension adjustment in the straps normally being done at the mask. The latch is easily attached and locked to the P&L fastener by sliding the latch onto the post. The latch can swivel 90 degrees or more without disengaging. It will not disengage when the strap connecting the mask to the fastener is slack. It is disengaged only by a deliberate action on the part of the diver. The fastener can easily be engaged or disengaged by a diver wearing gloves. The fastener prevents loss or dropping of the diver's mask in response to trauma such as being kicked in the mask by another diver's fin, being knocked off the top of the diver's head by a wave at the surface, removed by the force of impact while doing a giant stride entry, while diving, swimming through and becoming entangled in kelp, and the like.

### DRAWINGS—Figures

FIG. 1 is a side view of a diver's hood with a built-in, reinforced neoprene strip, post-and-latch fastener, and mask over the diver's eyes, in accordance with the invention.

FIG. 2 is a side view of the assembly of FIG. 1 with the mask on top of the diver's head.

FIG. 3 is a side view of the hood with a built-in, reinforced neoprene strip.

FIG. 4 is a cross-sectional view of the reinforced neoprene strip assembly.

FIG. 5 shows the layers of the reinforced neoprene strip.

FIG. 6 is a side view of an inner post used in the assembly of FIG. 1.

FIG. 6A is a cross-sectional view of an inner post used in the assembly of FIG. 1.

FIG. 7 is a side view of an outer post used in the assembly of FIG. 1.

FIG. 7A is a cross-sectional view of an outer post used in the assembly of FIG. 1.

FIG. 8 is an exploded view of a post assembly used in the assembly of FIG. 1.

FIG. 9 is a cross-sectional view showing the post assembly inserted in the neoprene strip assembly.

FIG. 10 shows a perspective view of a latch used in the assembly of FIG. 1.

FIG. 11 shows a perspective view of an alternative latch assembly.

FIG. 12 shows a cross-sectional view of the alternative latch and post.

FIG. 13 shows a cross-sectional view of the alternative latch positioned for release from an alternative post.

FIG. 14 shows a perspective view of an alternative latch and post design.

FIG. 15 shows a cross-sectional view of the alternative latch and post design.

FIG. 16 is an exploded view of an alternative post assembly.

FIG. 17 shows a cut-away view of the alternative post assembly assembled.

FIG. 18 shows a cut-away view of an alternative latch which is released by pressing, instead of linking the finger.

FIG. 19 shows a cut-away view of the alternative latch in place on an alternative post assembly.

FIG. 20 shows a cut-away view of the alternative latch with its release tab depressed

FIG. 21 shows a cut-away view of the alternative latch partially removed from an alternative post assembly.

FIG. 22 shows a top view of the alternative latch in place on a post on a diver's hood, holding the diver's mask in place on his or her eye area.

FIG. 23 shows a top view of the alternative latch still locked in place with the diver's mask on top of the diver's head.

FIG. 24 shows an exploded view of an alternative post assembly.

FIG. 25 shows a cross-sectional view of the assembled alternative post assembly. FIG. 26 shows a perspective view of an alternative latch and strap assembly.

FIG. 27 shows a cross-sectional view of the alternative latch and post assembly.

FIG. 27A shows a side view of an alternative strap.

FIG. 28 shows a side view of an alternative locking pin with a pointed tip.

FIG. 29 shows a cross-sectional view of an alternative locking pin with a pointed tip fully inserted into a post assembly.

FIG. 30 shows a section of neoprene attached to a strap that fits around the diver's neck in order to prevent loss of the diver's mask.

FIG. 31 shows the section-and-strap assembly in place on the diver's head and neck.

FIG. 32 shows an alternative retaining mechanism with a strap for securing the neoprene section to the diver's diving gear.

FIG. 33 shows a detail of an alternative, coiled retaining strap.

FIG. 34 shows the alternative retaining mechanism connected between the neoprene section on the diver's head to the diving gear.

FIG. 35 shows an alternative connection between the neoprene section and the strap in place on the diver's head.

FIG. 36 shows an alternative retaining mechanism for securely fastening a mask strap to the diver's hood.

FIG. 37 shows an alternative retaining mechanism for fastening a mask strap to the diver's hood using a H&L fastener.

DRAWINGS-Reference Numerals

| | | |
|---|---|---|
| 100 Mask | 420 Hole | 702 Base |
| 105 Strap | 425 Hole | 705 Column |
| 110 Latch | 430 Hole | 710 Flange |
| 115 Post | 600 Inner post | 715 Recesses |
| 120 Strip Assembly | 602 Base | 800 Locking pin |
| 125 Hood | 603 Points | 805 Base |
| 130 Seam | 605 Column | 810 Column |
| 400 Strip | 615 Ridge | 815 Flange |
| 405 Strip | 620 Hole | 900 Post assembly |
| 410 Strip | 625 Recesses | 1105 Strap adjuster |
| 415 Hole | 700 Outer post | 1110 Slot |
| 1115 Shoulder | 1730 Collar | 2560 Projections |
| 1120 Finger | 1735 Column | 2565 Recesses |
| 1122 Hook | 1740 Flange | 2567 Column |
| 1123 Inner edge | 1745 Recess | 2570 Cuts |
| 1124 Outer side | 1750 Head | 2572 Hood fabric |
| 1200 Latch | 1755 Locking pin | 2600 Post assembly |
| 1202 Larger portion | 1760 Recesses | 2640 Recess |
| 1203 Smaller portion | 1765 Ridge | 2680 Tapered inner edge |
| 1205 Post | 1800 Undercut | 2685 Tapered outer edge |
| 1210 Ramp | 1900 Latch | 2700 Latch assembly |
| 1215 Finger | 1905 Tab | 2705 Strap |
| 1216 Ramp | 1910 Finger | 2710 Finger |
| 1220 Hook | 2500 Post assembly | 2715 Hook |
| 1500 Latch | 2505 Locking pin | 2720 Latch |
| 1505 Finger | 2510 Column | 2810 Recess |
| 1510 Post | 2515 Flange | 2820 Ramp |
| 1515 Ramp | 2520 Recess | 2830 Slot |
| 1700 Post assembly | 2525 Point | 2840 Flange |
| 1705 Inner post | 2530 Upper flange | 2850 Short leg |
| 1710 Column | 2535 Outer post | 2900 Locking pin |
| 1715 Ridges | 2545 Column | 2905 Flange |
| 1720 Base | 2550 Ridge | 2910 Outer edge |
| 1725 Projections | 2555 Inner post | 2920 Recess |
| 3000 Post assembly | 3135 F. End of clasp | 3510 End |
| 3015 Outer post | 3140 End of strap | 3520 Head |
| 3020 Recess | 3145 H&L fastener | 3525 Strap |
| 3100 Head Strap | 3210 Latch | 3530 Stiching |
| 3110 Posts | 3300 Tether Strap | 3600 Strap |
| 3120 Strap | 3305 C-shaped clip | 3610 Plastic loop |
| 3125 Connecting strap | 3400 Coil Strap | 3700 Half H&L fastener |
| 3130 M. End of clasp | 3500 Hose | 3710 Half H&L fastener |

### DETAILED DESCRIPTION

### First Embodiment

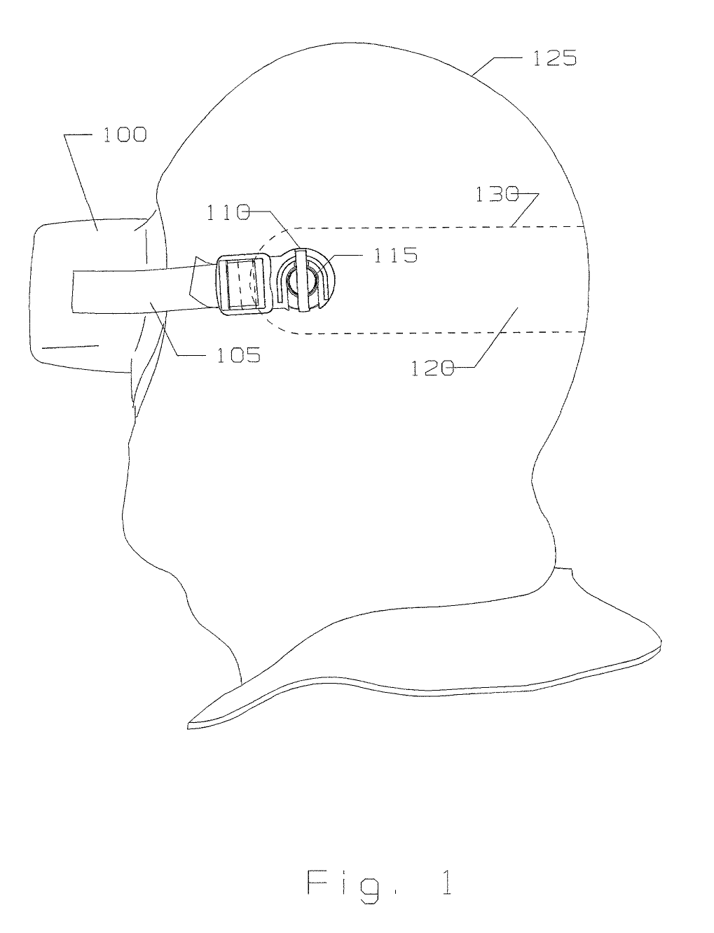

### Hood with Reinforced Strip and Swiveling, Locking Latch-and-Post Fasteners—Mask in Place Over Diver's Eyes—FIG. 1

A mask 100 is secured in an operative position to the face of a diver wearing a hood 125 by a strap 105, a latch 110, a post 115, and a reinforced neoprene strip assembly 120 attached to hood 125. Identical strip, latch, and post assemblies (not shown) are located on the opposite side of hood 125 in complementary positions. In the operative position, mask 100 protects the diver's eyes and nose, enabling underwater vision.

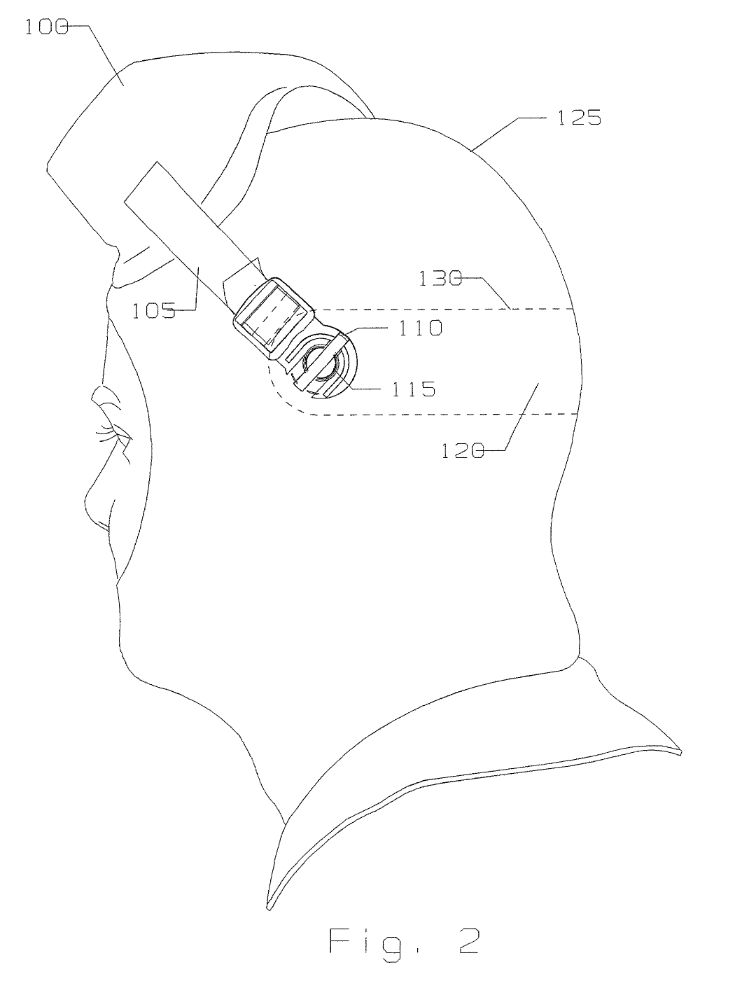

When viewed from the front, mask 100 is generally rectangular in shape, 20 cm wide, 10 cm high, and 7 cm deep. It covers the diver's eye and nose area (not shown), forming a seal around the perimeter bounded by the wearer's forehead, temples, and upper lip. Straps 105 attached to the sides of the mask terminate in latches 110 that attach to posts 115 affixed to the hood. Mask 100 is in a storage, inactive, inoperative, or carrying position when it is positioned on top of the diver's head (FIG. 2). Straps 105 communicating between mask 100 and latches 110 are generally made of sturdy, slightly stretchy silicone rubber. Hood strip assembly 120, generally made of reinforced neoprene, is discussed in detail below.

### FIG. 2—Mask Resting on Diver's Head

FIG. 2 shows mask 100 moved to its storage position on hood 125. In this position, the diver can breathe through their nose, has unobstructed vision, and can attend to various matters prior to entering the water, while floating or swimming on the surface of the water, or after leaving the water. Although mask 100 is removed from the diver's eyes, strap 105 is still securely anchored by latch 110 and post 115 and will not disengage. The matching strap, latch, and post on the opposite side of hood 125 are also anchored similarly. Thus mask 100 is firmly secured to straps 105 that terminate in latches 110 that attach to posts 115 affixed to the diver's hood 125.

To move mask 100 to the storage position top of his or her head, the diver simply grasps it, moves it forward, slightly stretching straps 105 to lift mask 100 away from his or her face, and rotates or pushes mask 100 upward. Latches 110 rotate on posts 115 without becoming detached. When mask 100 is released, it rests on top of hood 125.

### FIGS. 3-5—Reinforced Neoprene Strip

FIG. 3 shows a side view of hood 125 with seam 130 of strip assembly 120. Strip assembly 120 comprises three stitched and laminated strips, described in detail below. Strip assembly 120 can be built into the hood 125 with sewn seam 130 or constructed as a separate item and affixed to hood 125 by adhesive (not shown), a H&L fastener (not shown), or by sewn seam 130.

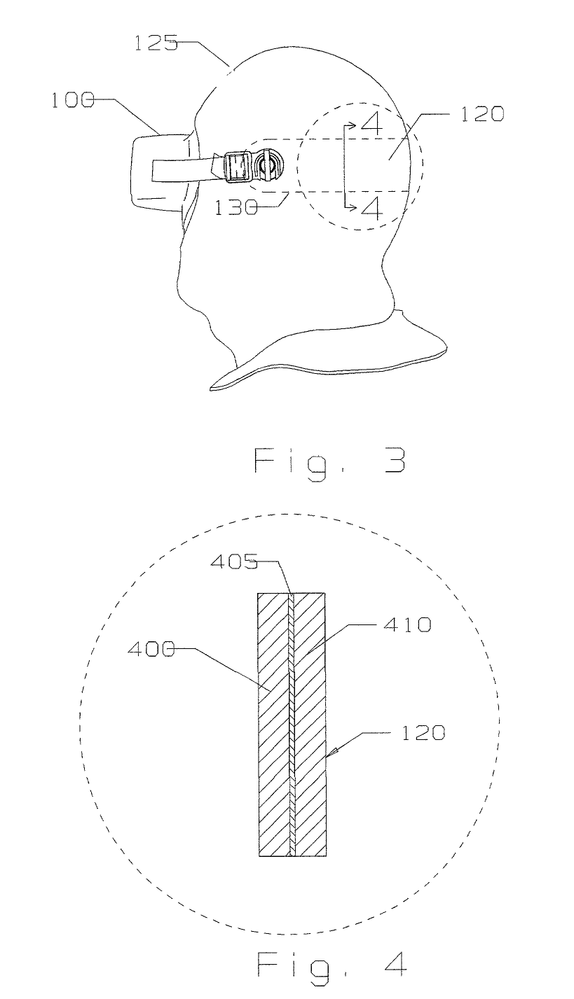

FIG. 4 shows a cross-sectional view of strip assembly 120. Strips 400, 405, and 410 preferably are bonded together by glue.

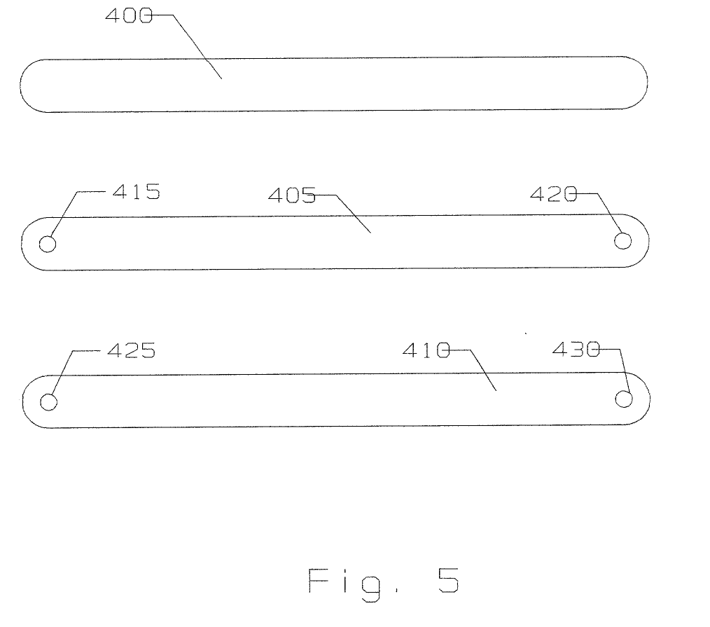

FIG. 5 shows the three strips that comprise strip assembly 120 (FIG. 4). All three have the same length and width, approximately 30.5 cm and 2.5 cm. A first strip 400 is preferably made of neoprene rubber, approximately 3 mm thick. A second strip 405 is made of nylon fabric, approximately 0.5 mm thick. Third strip 410 is also made of neoprene, approximately 3 mm thick.

Strips 405 and 410 contain holes 415, 420, 425, and 430, all about 8 mm in diameter. Strip 405 and 410 are stacked flat and glued together so that holes 415 and 425 are aligned with each other, and holes 420 and 430 are aligned with each other.

Strip 400 is added to the stack and glued after two inner posts 600 (FIG. 6) are inserted through holes 415 and 425, and 420 and 430.

### FIGS. 6-9—Post Assembly

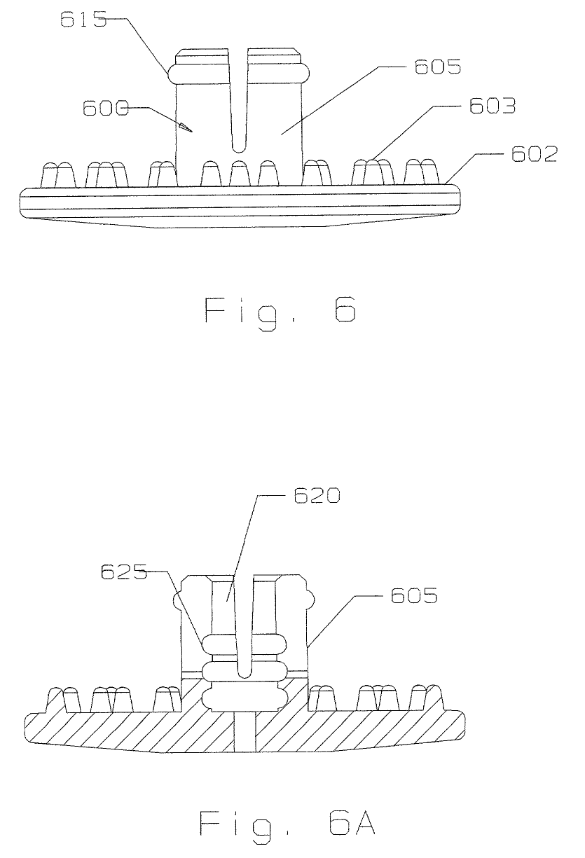

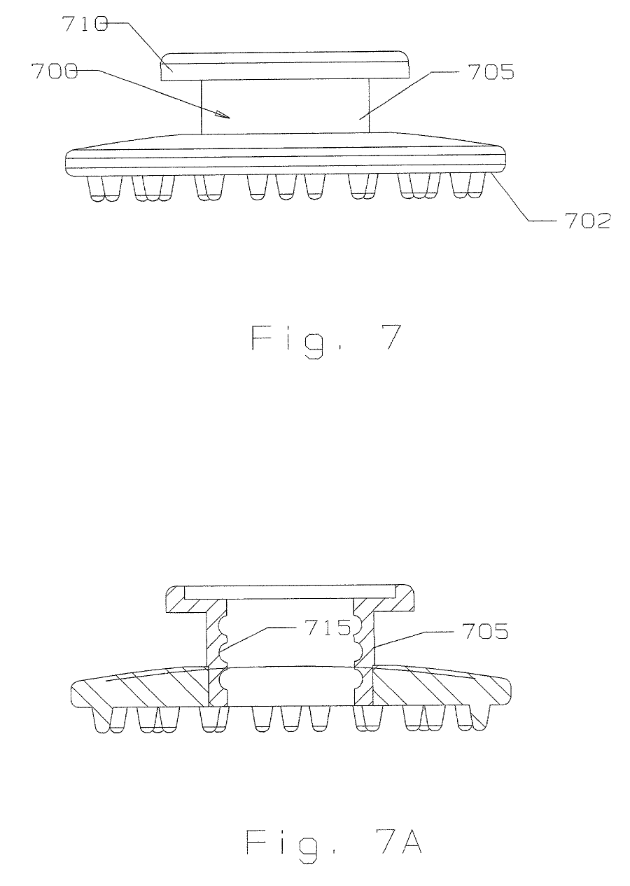

Post 115 (FIGS. 1 and 2) comprises three components. From the outside of hood 125 (FIG. 1), post 115 appears as a column resting on a circular disc. The top of the column terminates in a flange, slightly larger in diameter than the column. Post 115 comprises an inner post 600 (FIG. 6), located inside hood 125, an outer post 700 (FIG. 7), located on the outside of hood 125 and coupled with inner post 600, and a locking pin 800 (FIG. 8) which rigidly fixes inner post 600 and outer post 700 together as a unit.

FIG. 6 shows a side view of an inner post 600. Base 602 of inner post 600 is approximately 2.5 cm in diameter. Base 602 has a series of raised points 603 that are preferably 2 mm in diameter and 2 mm high. Inner post 600 has a central column 605 with four slots extending partway from the distal end toward the base. Central column 605 is preferably about 7.6 mm in diameter, and 8.1 mm high, with a ridge 615 near the top. Ridge 615 is preferably half-round in contour and about 8.8 mm in diameter.

FIG. 6A shows a cross-sectional view of inner post 600 (FIG. 6). A hole 620, about 3.9 mm in diameter, extends through column 605. Column 605 preferably contains three internal recesses 625. These recesses are semicircular in shape and have a maximum diameter of 5 mm.

FIG. 7 shows a side view of an outer post 700. Base 702 of outer post 700 is about 2.5 cm in diameter. Outer post 700 has a central column 705 about 4.3 mm high, with an inner diameter of 7.6 mm and an outer diameter of 10.1 mm. At one end, column 705 is joined with base 702. At the other end, column 705 terminates in a flange 710. The outer diameter of flange 710 is preferably 14.9 mm.

FIG. 7A shows a cross-sectional view of outer post 700 (FIG. 7). The inner surface of column 705 preferably has three recesses 715, each with a diameter of 8.8 mm.

FIG. 8 shows a locking pin 800 and all the components of post assembly 900. Locking pin 800 has a base 805 with diameter of approximately 12.4 mm and height 1.4 mm. Column 810 is axially joined to base 805. The diameter of column 810 is preferably 3.9 mm. Column 810 terminates in a flange 815 with an outer diameter 5.0 mm.

The components of post assembly 900 comprising inner post 600, outer post 700, and locking pin 800 join together in the order shown in FIG. 8. Column 605 of inner post 600 is passed through holes 415 and 425 or 420 and 430 (FIG. 5) of strip assembly 120 (FIG. 9). Strips 405 and 410 (FIG. 9) are compressed between the top of base 602 of inner post 600 and the bottom of base 702 of outer post 700.

Ridge 615 on column 605 of inner post 600 springably enters one of the recesses 715 in column 705 of outer post 700. Inner post 600 and outer post 700 are forcibly mated, moving ridge 615 as far into column 705 as possible. Thus ridge 615 expands into the innermost possible recesses of column 705, temporarily securing inner post 600 and outer post 700 on strip assembly 120 (FIG. 9).

Next, locking pin 800 is inserted into the inner opening of column 605. There, flange 815 of locking pin 800 forcibly enters one of the recesses 625 (FIG. 6A) in column 605 of inner post 600. Locking pin 800 is inserted as far as possible into column 605, expanding into recess 625 (FIG. 6A), as determined by the location of inner post 600 with respect to outer post 700. With these conditions met, inner post 600, outer post 700, and locking pin 800 will be securely fastened to strip assembly 120 (FIG. 9).

FIG. 9 shows a cross-sectional view of post assembly 900 in place in strip assembly 120.

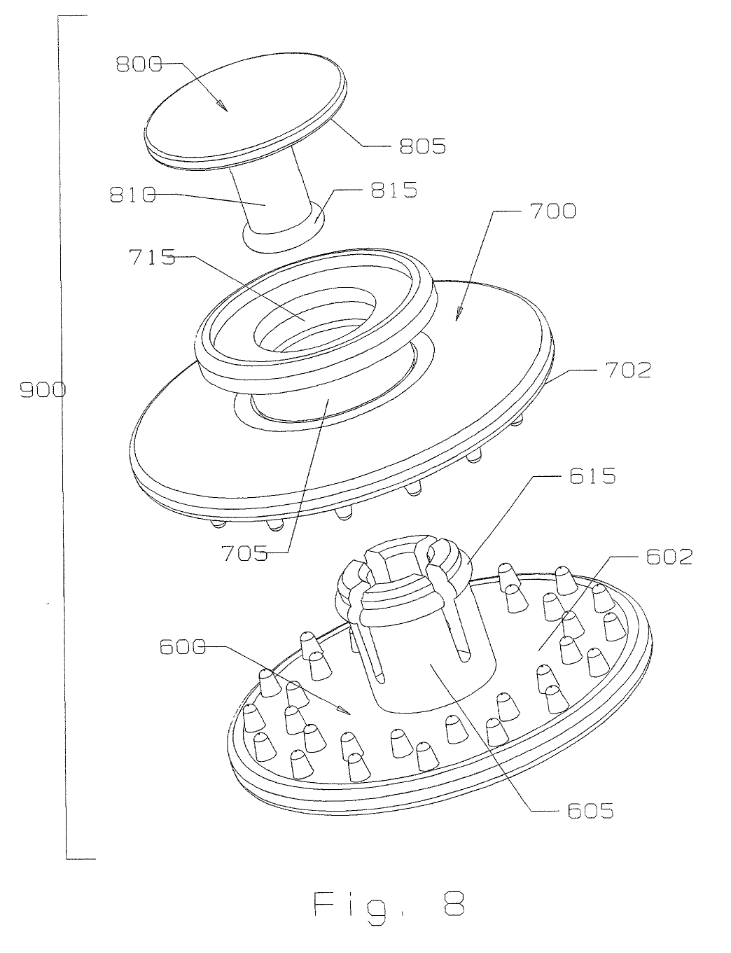

### FIG. 10—Locking Latch

FIG. 10 is a perspective view of a locking latch 110. A strap adjuster 1105 is attached to latch 10. Instead, strap adjuster 1105 can be replaced with an attachment (not shown), which permanently affixes a strap (not shown) to latch 110.

Latch 110 comprises a slot 1110 of width approximately 12 mm and depth about 14 mm, a shoulder 1115 with inner width about 15 mm and depth about 17 mm, and a hooked finger 1120. Finger 1120 is about 6 mm wide and 18 mm long. Hook 1122 on finger 1120 extends approximately 2.5 mm downward toward slot 1110. The plane forming the inner edge 1123 of hook 1122 is perpendicular to the bottom surface of finger 1120. The outer side 1124 of hook 1122 is angled to form a ramp for sliding over flange 710 of outer post 700 (FIG. 7).

In use, strap 105 (FIG. 1) is affixed to latch 110, then to one side of mask 100 (FIG. 1). As described above in connection with FIG. 1, a complimentary strap and latch are used on the opposite side of the diver's head.

Latch 110 is attached to post assembly 900 (FIG. 8) by simply sliding slot 1110 over column 705 of outer post 700 (FIG. 7). The underside of flange 710 of outer post 700 rests on shoulder 1115, preventing latch 110 from moving in a direction away from base 702 of outer post 700. The underside of finger 1120 rests on the top of locking pin 800 (FIG. 8), preventing latch 110 from moving in a direction toward the base 702 of outer post 700. Column 705 of outer post 700 rests against the inner, curved end of slot 1110, preventing latch 110 from moving in a direction toward column 705 of outer post 700. Finger 1120 straddles the top of flange 710, while inside edge 1123 of hook 1122 rests against the outer side of flange 710. Slot 1110, shoulder 1115, finger 1120, and hook 1122 prevent any relative motion of latch 110 and outer post 700. Thus latch 110 is securely attached to outer post 700.

To remove latch 110 from outer post 700 (FIG. 7), the wearer simply lifts hook 1122 of finger 1120, bending finger 1120 so that the bottom of hook 1122 is raised above the tops of flange 710 and locking pin 800 (FIG. 8). Latch 11O can then slide off of outer post 700. The lifting of hook 1122 of finger 1120 must be done by a deliberate action on the part of the diver. Thus the mask will detach from the hood only when the diver desires.

### Operation—FIGS. 1-10

Latch 110 engages outer post 700 by sliding over it until locked in place by hook 1122 of finger 1120. Latch 110 is released by lifting finger 1120 and sliding latch 110 off outer post 700. This assembly provides a latch that is secure and that can be disengaged only by a deliberate action of the diver.

### Second Embodiment

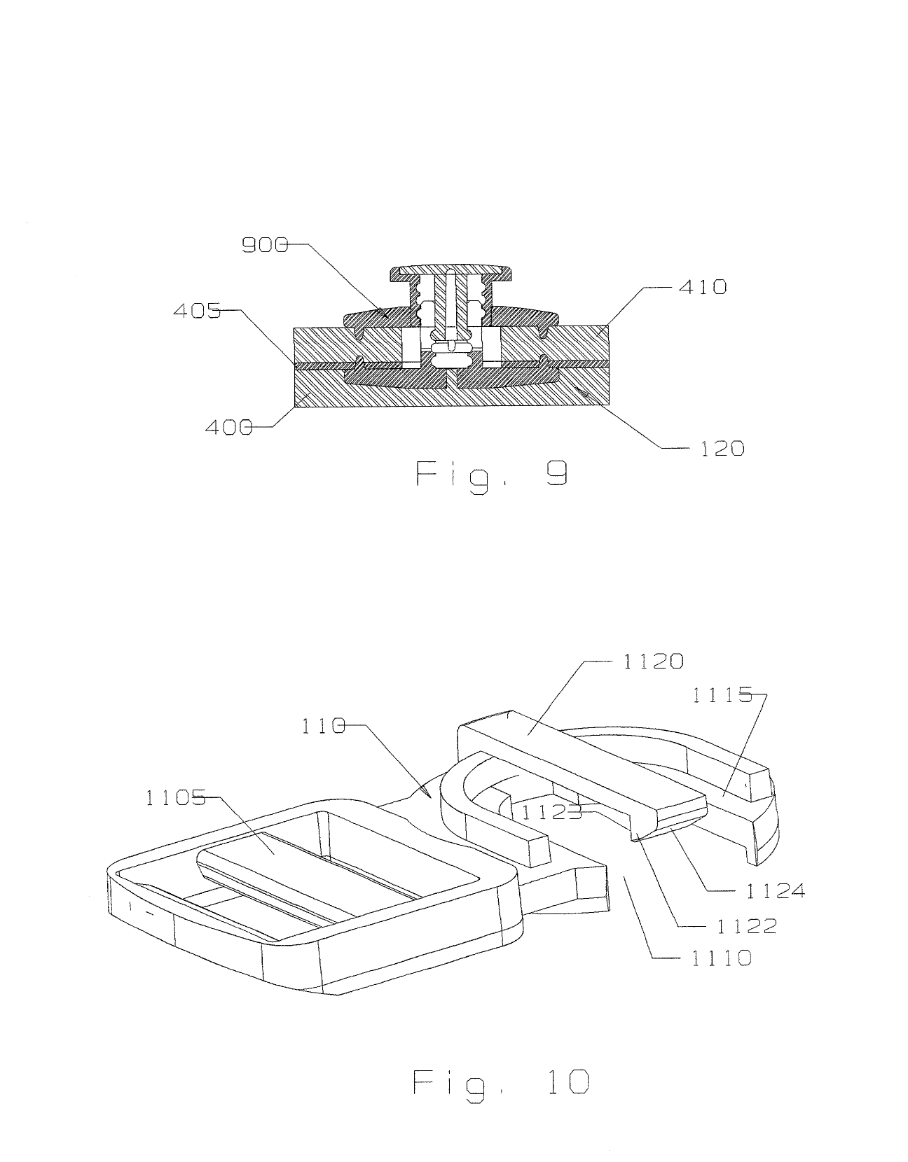

### Covered Locking Latch with Swivel Release Feature—FIGS. 11-13

In this and all subsequent embodiments, dimensions of the post and latch are similar to those of the first embodiment.

This embodiment comprises a covered slotted fastener with a locking finger. The inner post and locking pin are the same as in the first embodiment. The outer post is also the same, except for the addition of a ramp on one side.

FIG. 11 shows a perspective view of a covered latch 1200. To secure latch 1200, the wearer inserts post 1205 (FIG. 12) into the larger portion 1202 of the slotted fastener comprising larger portion 1202 and smaller portion 1203. The shape of the opening in latch 1200 is similar to that shown in FIG. 26 below. When post 1205 is fully inserted in larger portion 1202, latch 1200 is slid over post 1205 so that post 1205 moves into smaller portion 1203 of the slotted fastener. Ramp 1216 (FIG. 12), lifts finger 1215 (FIG. 12) on the way in.

As shown in FIG. 12, when post 1205 is fully inserted into smaller portion 1203, finger 1215 rests on post 1205 and hook 1220 on finger 1215 blocks the exit of post 1205.

Latch 1200 can be removed from post 1205 in two ways. Finger 1215 can be lifted so that hook 1220 is raised above the top of post 1205, enabling latch 1200 to be removed from post 1205 as described above. Alternatively, latch 1200 can be rotated so that hook 1220 is aligned adjacent to ramp 1210.

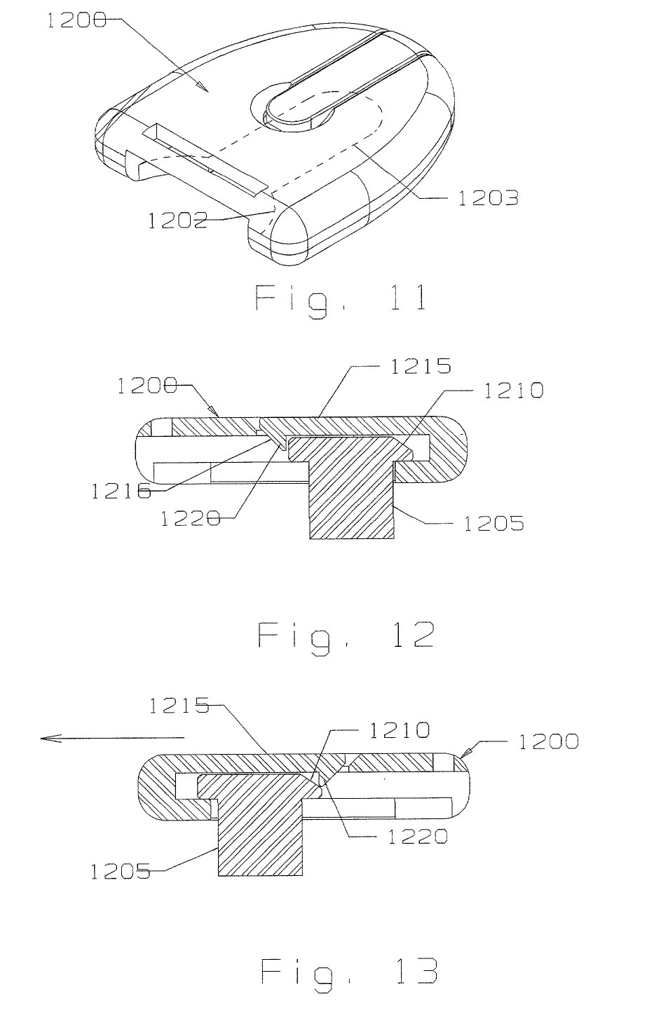

FIG. 13 shows hook 1220 engaged against and partially up ramp 1210. If the user continues to push latch 1200 in the direction of ramp 1210, latch 1200 wire disengage from post 1205.

Once again, latch 1200 is securely attached to post 1205. Only a deliberate action of the user can separate the two parts. Either finger 1215 must be raised, or latch 1200 must be rotated 180 degrees in order to slide latch 1200 away from ramped post 1205. Because latch 1200 has a smooth cover, and because latch 1200 covers post 1205, this design is virtually immune to snagging on kelp and the like.

### Operation—FIGS. 11-13

Latch 1200 engages post 1205 by sliding over it until hook 1220 on finger 1215 falls on the trailing edge of post 1205. Latch 1200 is securely fastened to post 1205 until deliberately removed by lifting finger 1215 and sliding latch 1200 off post 1205. Alternatively, the diver can rotate latch 1200 to a position at which hook 1220 of finger 1215 can slide up ramp 1210, allowing latch 1200 to slide off post 1205. Thus in this embodiment, latch 1200 can be removed from post 1205 in either of two ways.

### Third Embodiment

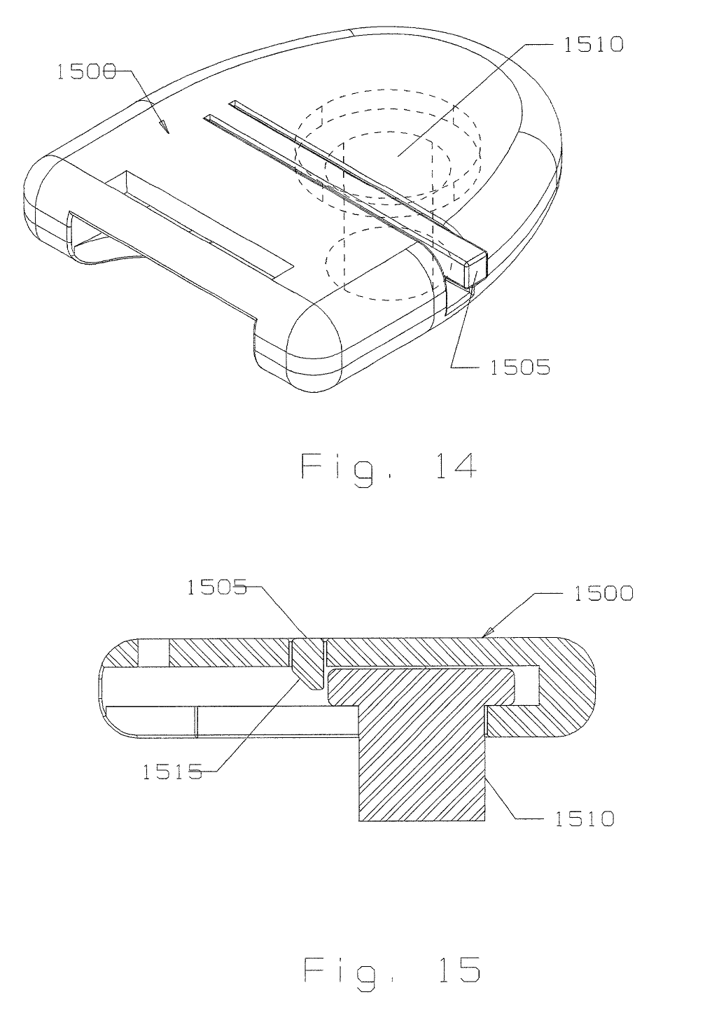

### Alternative Release Finger Position—FIGS. 14-15

FIG. 14 shows a perspective view of a modified latch 1500 with release finger 1505 in a position adjacent post 1510, instead of straddling it. The inner and outer posts and locking pin are the same as in the first preferred embodiment. The latch is similar to that in the second embodiment, except that the finger is relocated to allow 360 degrees of latch engagement.

FIG. 15 shows a cross-sectional, side view of latch 1500 and post 1510 in the locked position. Finger 1505 has a ramp 1515 to permit the user to slide latch 1500 over post 1510 during engagement. Ramp 1515 lifts finger 1505 as post 1510 is moved into the locked position in latch 1500.

Latch 1500 is fly attached to post 1510 and can be removed only by raising finger 1505.

### Operation—FIGS. 14-15

Latch 1500 is slidably engaged with post 1510 and is securely locked when finger 1505 falls on the trailing edge of post 1510. Post 1510 enters latch 1500 from the end nearest the point at which the strap (not shown) is attached to latch 1500. Latch 1500 is disengaged by lifting the end of finger 1505, located on the side of latch 1500 instead of its end. Thus this embodiment provides a different position for entry and exit of post 1510, and a different position for finger 1505.

### Fourth Embodiment

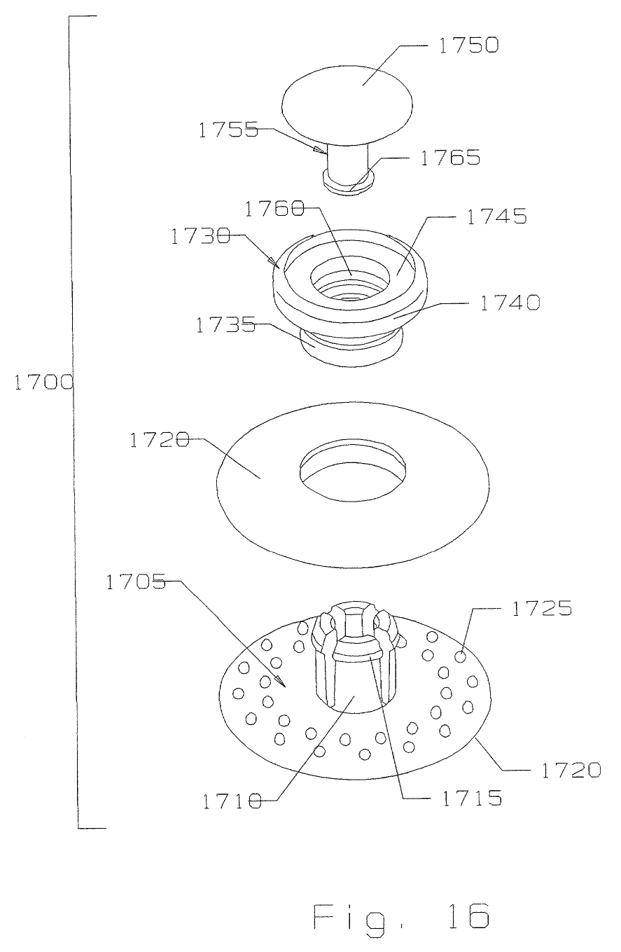

### Alternative Release Method—FIGS. 16-23

In this embodiment, the inner post is the same as that used in the first embodiment above. The outer post is the same as in the first embodiment, except that it contains an undercut on the bottom of its flange for the finger to hook into. The locking pin is the same as that shown in the first embodiment, except that relief slots have been cut into its shaft. The latch is the same as that shown in the second embodiment above except that the finger hooks into the undercut on the bottom of the flange of the outer post. This allows 360 degrees of latch engagement. In this embodiment, the latch and post assembly is separated by pressing downward on a lever, instead of lifting a finger.

FIG. 16 shows an exploded view of post assembly 1700, which comprises an inner post 1705 with split column 1710 having ridges 1715 at the top. Base 1720 of inner post 1705 contains numerous projections 1725 that help to anchor assembly 1700 in the hood strip assembly 120 (FIG. 1), or directly in the hood itself, as shown below.

Instead of being part of the outer post as shown in the above embodiments, collar 1730 is a separate component. Collar 1730 comprises a column 1735, and a flange 1740. Collar 1730 has a recess 1745 in the top to accommodate head 1750 of locking pin 1755. Collar 1730 has a number of inner recesses 1760 (three are shown, although there can be more or less). Recesses 1760 accepts ridge 1715 of inner post 1705, in the same manner as in FIG. 8. When post assembly 1700 is assembled, collar 1730 slidably fits into base 1720.

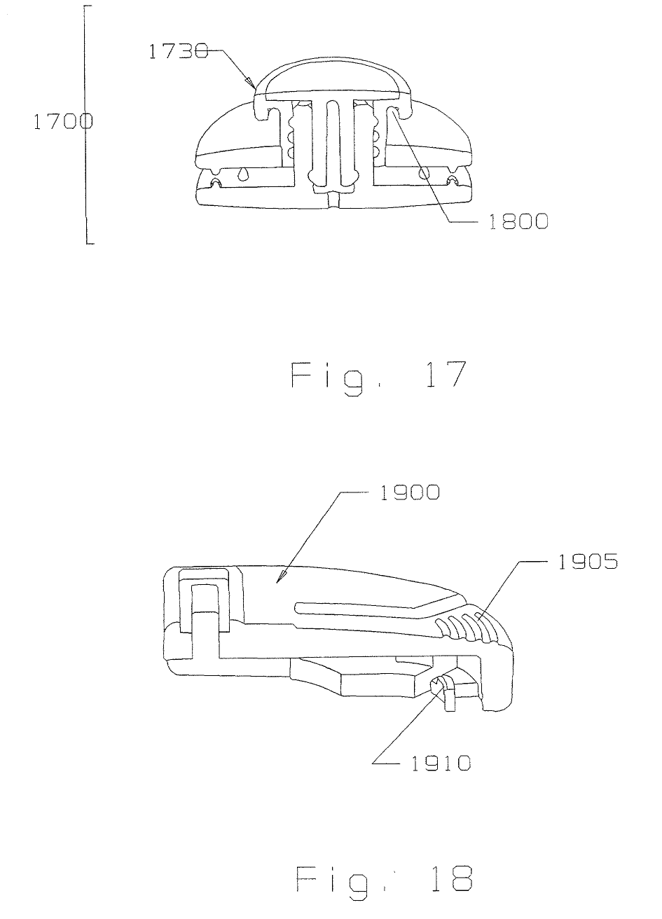

FIG. 17 shows a cut-away view of post assembly 1700 in the filly assembled condition. An undercut 1800 in collar 1730 accepts a locking finger, as described below in connection with latch 1900 (FIG. 18).

FIG. 18 is a cut-away view of latch 1900. Latch 1900 contains a tab 1905, to which is attached a finger 1910. When latch 1900 is secured to post assembly 1700, finger 1910 resides in undercut 1800. While engaged to post assembly 1700, latch 1900 can swivel 360 degrees without becoming disengaged. Finger 1910 is free to follow the circular path within the channel created by undercut 1800.

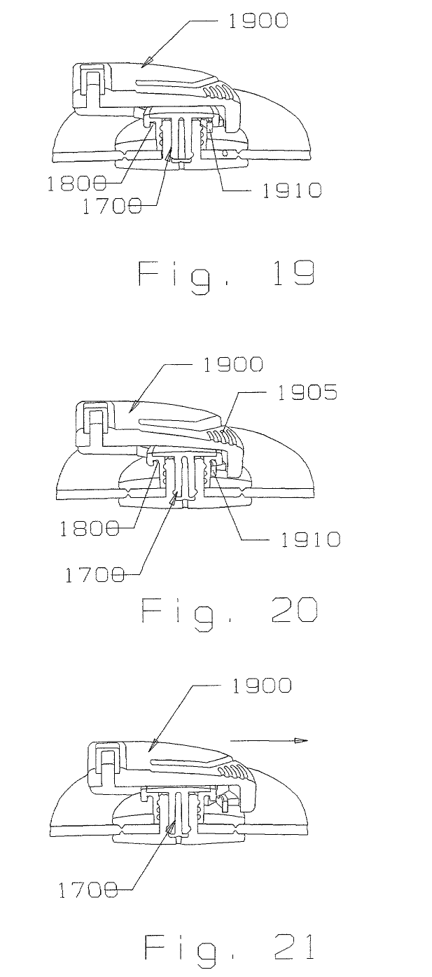

FIG. 19 shows latch 1900 locked on post assembly 1700. Finger 1910 is ally inserted into undercut 1800.

Tab 1905 of latch 1900 is shown in the depressed position in FIG. 20. Finger 1910 is removed from undercut 1800. This permits latch 1900 to slidably disengage from post assembly 1700.

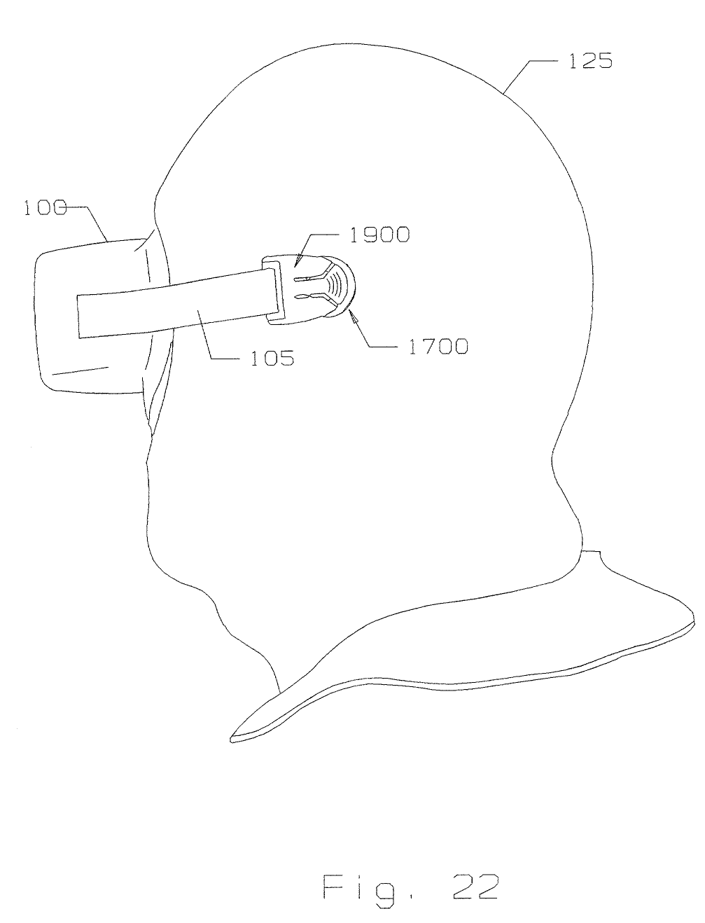

FIG. 21 shows latch 1900 being removed from post assembly 1700.

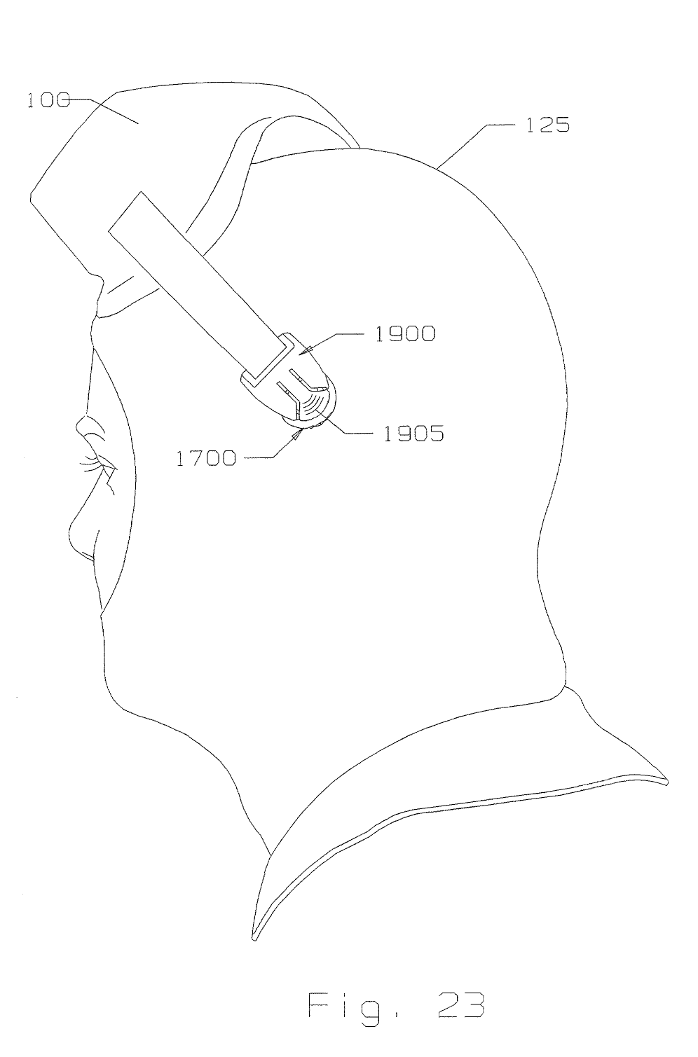

Mask 100 is secured over the diver's eyes by strap 105 (FIG. 22). Strap 105 is attached to mask 100 and latches 1900 (on each side of mask 100), and post assemblies 1700 on hood 125. Post assembly 1700 is shown directly attached to hood 125, without the use of strip assembly 120 (Fig 3).

FIG. 23 shows mask 100 still securely attached to hood 125, but in a raised position on top of the diver's head.

Again, this latch-and-post fastener is free to swivel, yet cannot be disengaged without deliberate action on the part of the wearer. In this case, tab 1905 must be depressed and latch 1900 slid off post assembly 1700.

### Operation—FIGS. 16-23

Post assembly 1700 slidably enters latch 1900 from the rear, or strap side, and securely locks when finger 1910 engages undercut 1800. Latch 1900 is disengaged by pressing tab 1905 to release finger 1910 from undercut 1800, then sliding latch 1900 off of post assembly 1700. Thus in this embodiment, latch 1900 is removed by the application of force in the direction of post assembly 1700, instead of away from it as in the embodiments described above.

### Fifth Embodiment

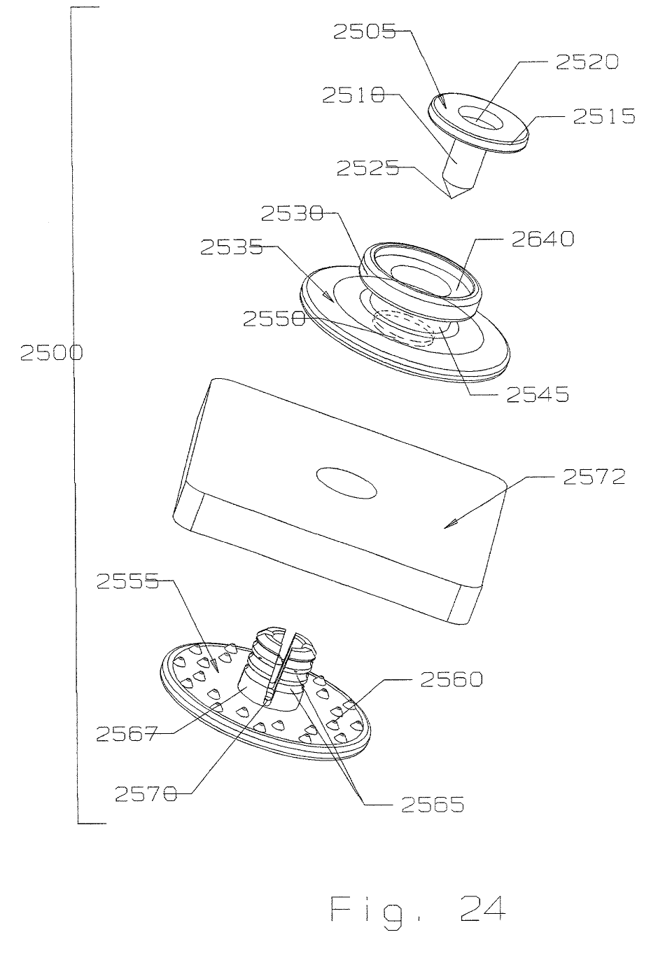

### Latch with Central Finger and Alternative Post Assembly—FIGS. 24-27A

In this embodiment, inner post 2555 and outer post 2535 slidably couple and are held in place by ridge 2550 on outer post 2535 and one of recesses 2565 on inner post 2555. A sharp point 2525 on locking pin 2505 enables installation of post assembly 2500 without the need to punch a hole in hood fabric 2572 of hood 125 (FIG. 1). Instead of a finger that latches at the perimeter of the upper flange 2530 on outer post 2535, hook 2715 of latch assembly 2700 engages recess 2520 in locking pin 2505.

FIG. 24 shows an exploded view of post assembly 2500. Locking pin 2505 has a smooth cylindrical column 2510 extending from flange 2515. Locking pin 2505 also has a circular recess 2520 in the center of flange 2515. Locking pin 2505 has a sharp point 2525 to facilitate installation on a hood 125 (FIG. 1), without the need to punch holes prior to installation.

Upper flange 2530 of outer post 2535 contains a recess 2640 which will be described in detail below. Central column 2545 contains an inner ridge 2550, which will also be described in detail below.

Both inner post 2555 and outer post 2535 contain numerous projections 2560 to more firmly secure post assembly 2500 to hood 125 (FIG. 1). Inner post 2555 also contains a number of recesses 2565 along column 2567. It also has cuts 2570 along column 2567 at several locations in order to facilitate assembly to outer post 2535.

Inner post 2555 and outer post 2535 are shown on either side of hood fabric 2572. Hood fabric 2572 is typically neoprene layered with nylon or another strengthening material

FIG. 25 shows a cross-sectional view of post assembly 2600 as assembled on a hood fabric of medium thickness. Hood fabric 2572 (FIG. 24) has been omitted from the drawing for clarity. Recesses 2565 in inner post 2555 mate with ridge 2550 on outer post 2535. Outer post 2535 can be slid over inner post 2555 because cuts 2570 (FIG. 24) at locations permit radial flexing of column 2567 (FIG. 24). Recesses 2565 act like detents for ridge 2550.

In the absence of locking pin 2505, outer post 2535 is able to slide on column 2567 (FIG. 24) with only the detent action supplied by recesses 2565 and ridge 2550. With locking pin 2505 inserted, the slotted column 2567 can no longer bend as ridge 2550 is moved up and down. Thus inner post 2555 and outer post 2535 are secured together.

Recess 2640 on upper flange 2530 of outer post 2535 has inward-sloping, tapered inner edge 2680. These mate with inward sloping tapered outer edge 2685 on flange 2515 of locking pin 2505. Thus once locking pin 2505 is filly inserted in outer post 2535; post assembly 2600 is firmly locked in place.

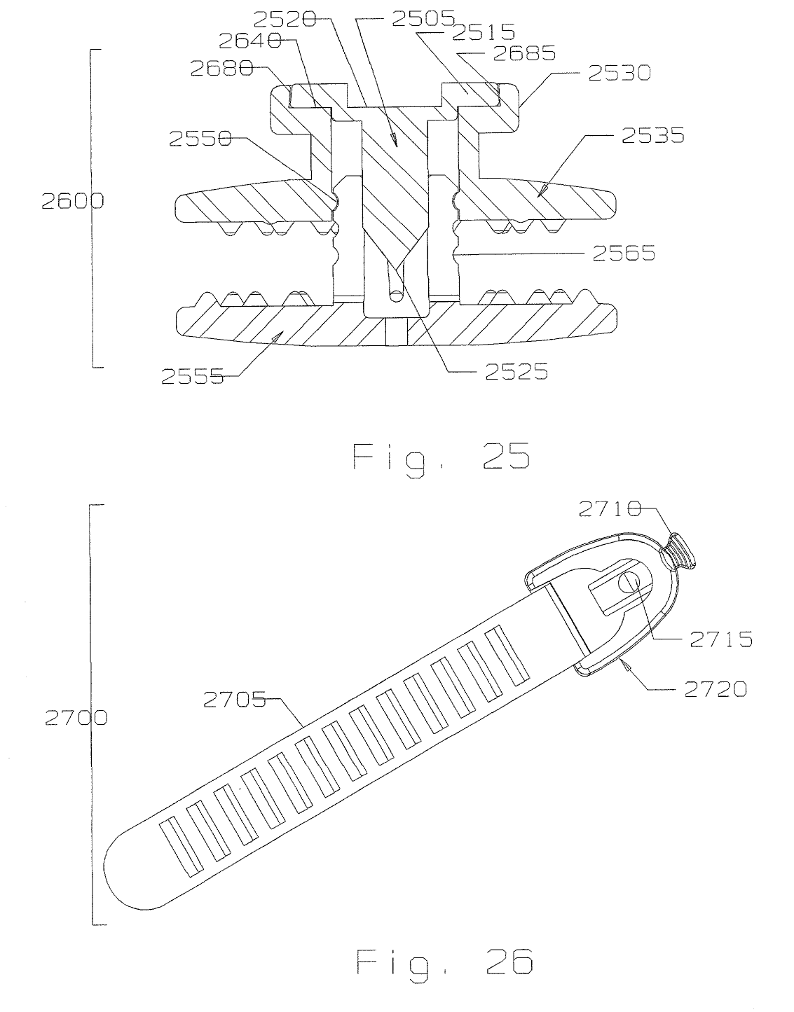

FIG. 26 shows a latch assembly 2700 designed for use with post assembly 2500. A strap 2705 is shown attached to latch 2720. Latch 2720 contains a finger 2710 with a hook 2715. Hook 2715 has a ramp 2820 (FIG. 27) on one side to facilitate mating latch 2720 with post assembly 2500.

FIG. 27 shows latch 2720 locked in place on post assembly 2500. To assemble the two, latch 2720 is simply slid over post assembly 2500. To separate the two, finger 2710 is lifted, removing hook 2715 from recess 2520 (FIG. 25) in locking pin 2505, allowing latch 2720 to be slid off of post assembly 2500.

FIG. 27A shows a side view of strap 2705. Strap 2705 is about 14 cm long, 20 mm wide and 2 mm thick. One end of strap 2705 has a short leg 2850 with a flange 2840 on top. The short leg 2850 extends 2.4 mm from strap 2705 to bottom of flange 2840. Short leg 2850 is 20 mm wide and 3.8 mm thick. Flange 2840 is rectangular in shape with a length, width, and depth of 6.6, 22, and 2 mm respectively.

Strap 2705 is inserted through slot 2830 in latch 2720 until the bottom of flange 2840 terminates in recess 2810. In this position strap 2705 exits the latch 2720 away from post assembly 2500 and towards mask 100 (FIG. 1). Flange 2840 prevents strap 2705 from pulling through slot 2830 in latch 2720. This allows tensioning of strap 2705 when attached to mask 100 (FIG. 1) and post assembly 2500.

### Operation—FIGS. 24-27A

When latch assembly 2700 and post assembly 2500 are fully engaged, hook 2715 enters recess 2520, securely locking latch assembly 2700 and post assembly 2500 together. This assembly can be swiveled 360 degrees without becoming disengaged. The diver must deliberately lift finger 2710 to disengage latch assembly 2700 from post assembly 2500.

### Sixth Embodiment

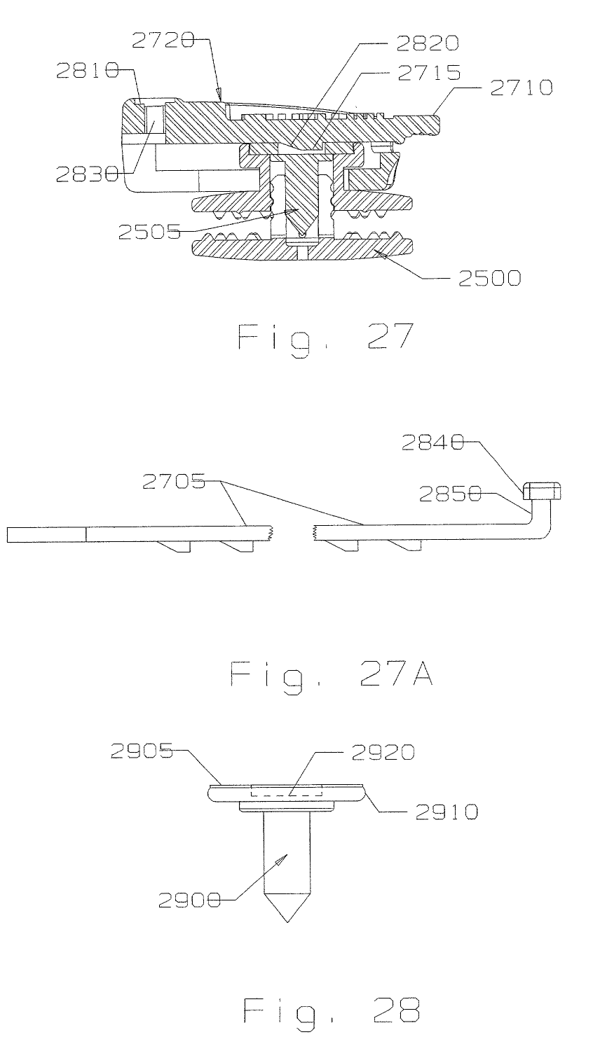

### Alternative Locking Pin Capture—FIGS. 28-29

The embodiment in FIG. 28 is similar to the one described above, except that the flange on the locking pin has a different shape. Flange 2905 on locking pin 2900 has a convex, rounded outer edge 2910.

FIG. 29 shows post assembly 3000 assembled with locking pin 2900. The top, inner edge of outer post 3015 contains an inner recess 3020 which mates with outer edge 2910 of locking pin 2900 to secure locking pin 2900. When locking pin 2900 is fully inserted in outer post 3015, post assembly 3000 is fully locked in place. Recess 2920 accepts hook 2715 (FIG. 27) of latch assembly 2700 (FIG. 26).

### Operation—FIGS. 28-29

Like all those described above, this assembly locks securely, can be rotated 360 degrees, and can be disengaged only through a deliberate action by the diver.

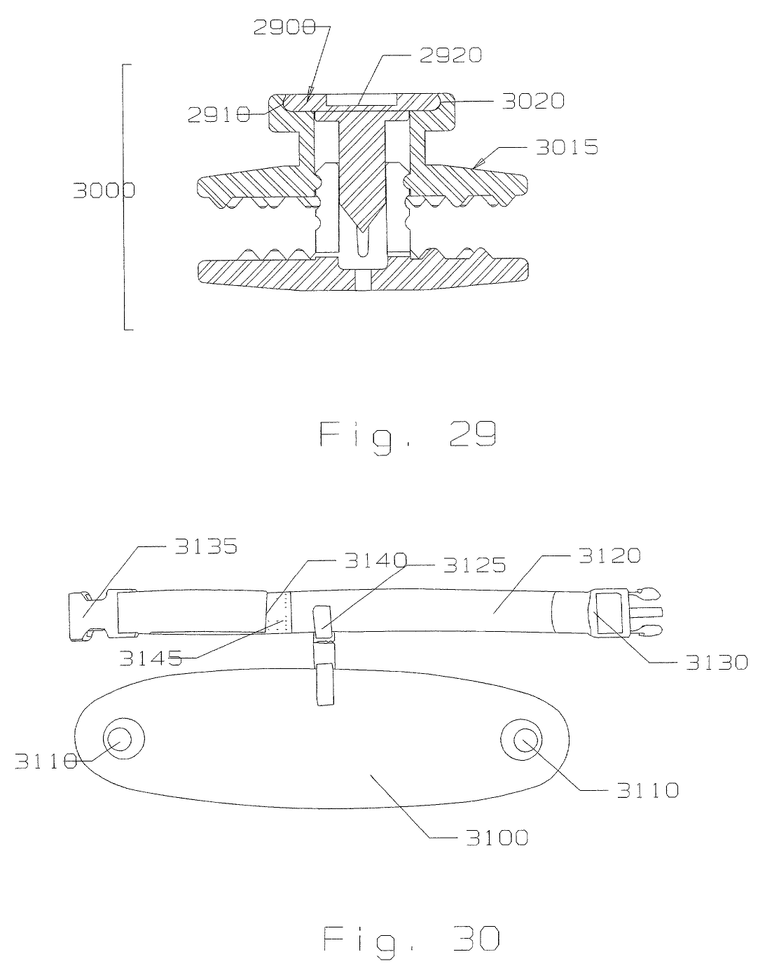

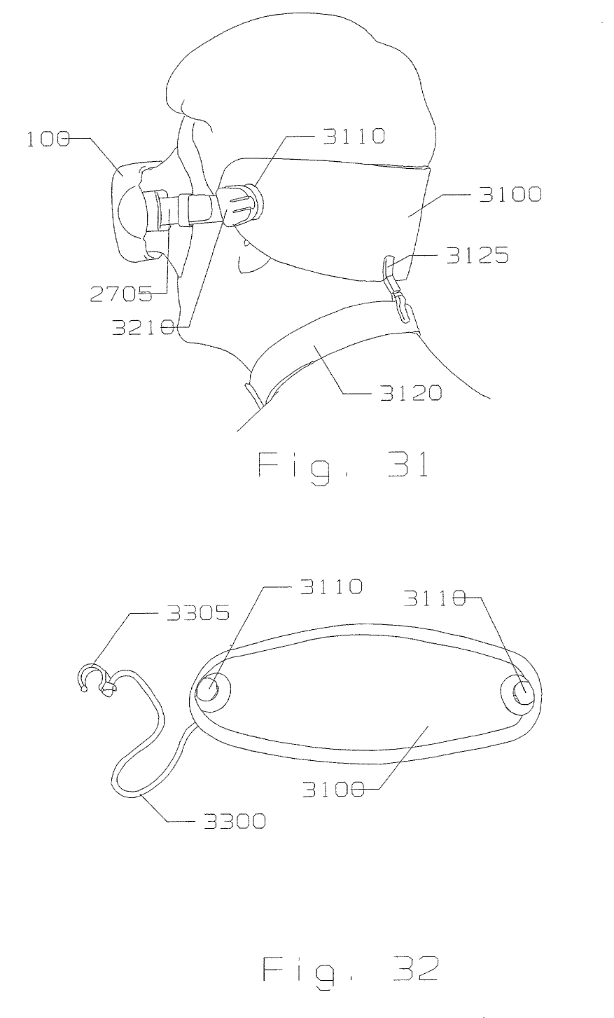

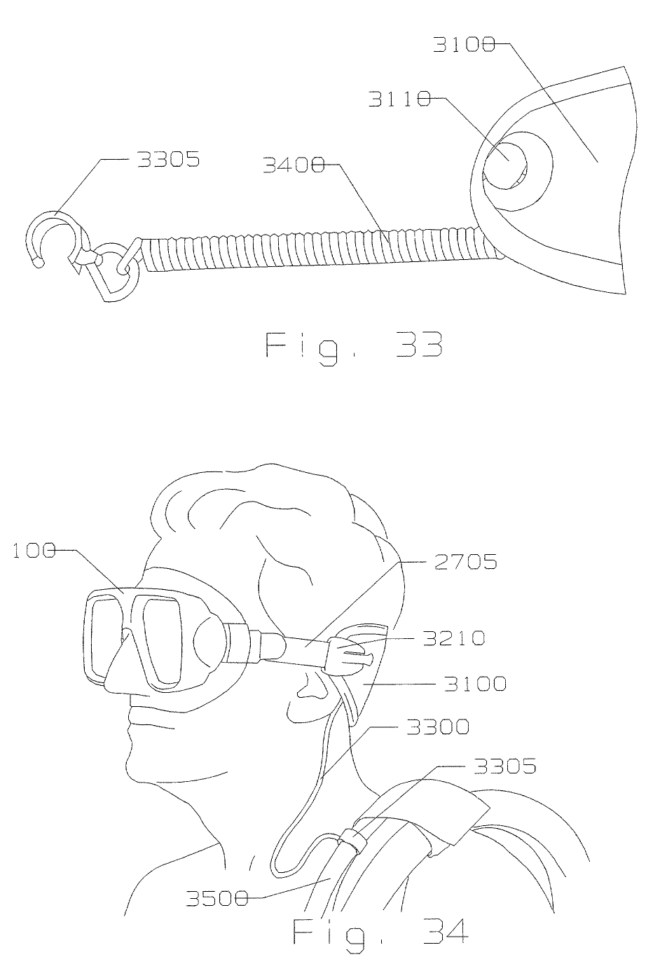

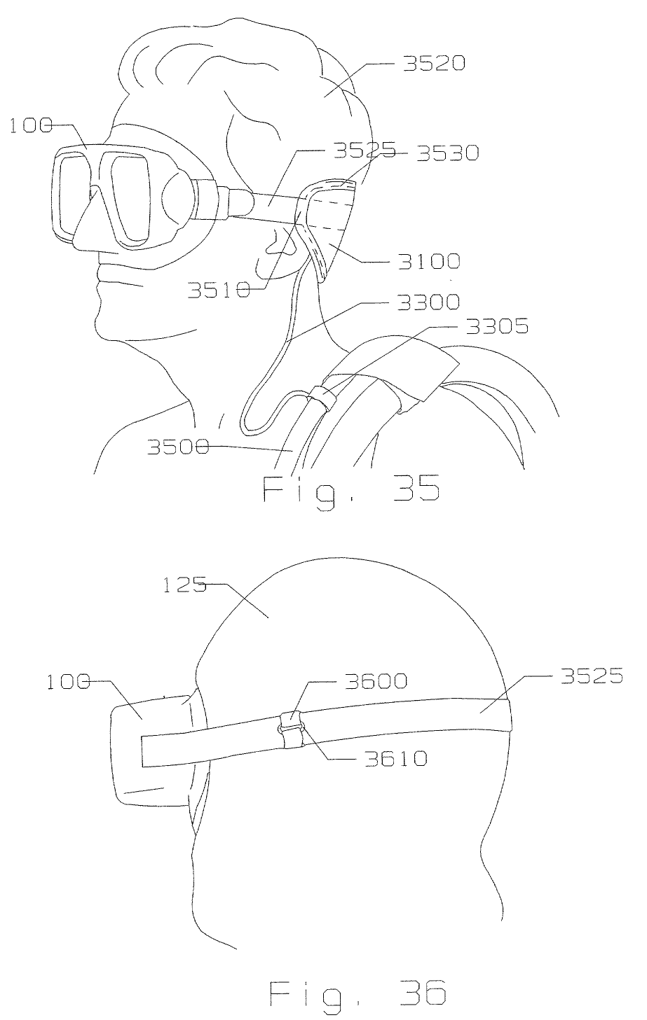

### Seventh Embodiment —Alternate, Captive Mask Mountings—FIGS. 30-35

In the above embodiments, the diver's mask is attached to posts that are secured to a hood that fits over and covers most of the diver's head. This hood is part of an exposure suit that keeps the diver warm in cold water. In warm-water diving, the exposure suit and hood are not always used. Therefore, another method is used to secure the mask to the diver.

FIG. 30 shows an oblong head strap 3100, preferably made of a neoprene. Strap 3100 is typically 30 cm long and 10 cm wide. Posts 3110 are secured to strap 3100 in the positions shown, typically 3 cm from each end.

A retaining or neck strap 3120 is attached to strap 3100 by a second, small connecting strap 3125. Neck strap 3120 is typically 40 cm long and 2.5 cm wide, and made of neoprene. Connecting strap 3125 is typically 2 cm long and 1 cm wide. A clasp, comprising male end of clasp 3130 and female end of clasp 3135 is fitted to the ends of neck strap 3120. The clasp can also be replaced with mating parts of H&L fastener, in well-known fashion to secure neck strap 3120. The length of neck strap 3120 is adjusted by positioning an end 3140 of the neck strap along a region containing mating parts of H&L fastener 3145, in well-known fashion.

One end of the connecting strap 3125, which may also be of adjustable length, is attached to the outer edge, center of head strap 3100. The other end of connecting strap 3125 is attached to neck strap 3120 near its center. Thus connecting strap 3125 locks head strap 3100 and neck strap 3120 together at their respective centers. Connecting strap 3125 can permanently connect head strap 3100 and neck trap 3120 together, or alternatively connecting strap 3125 can comprise a clamping arrangement that will release at a predetermined pulling stress. Releasing at a predetermined pulling stress is desirable in the event the mask gets entangled in seaweed, for example.

FIG. 31 shows head strap 3100 and connecting strap 3125 in place on the diver's head and neck, respectively. Neck strap 3120 surrounds the diver's neck and is secured in place by male and female ends 3130 and 3135 of clasp components (FIG. 30), respectively.

Straps 2705 from mask 100 terminate in latches 3210. Latches 3210 are described in detail above.

With this arrangement the diver is able to place mask 100 on top of his or her head with little fear that mask 100 will be lost. Even if mask 100 is displaced from the diver's eye area while diving or if it falls off the diver's head while floating or swimming at the surface, it will not be lost since it is securely attached to straps 2705, latches 3210, and posts 3110. The latter are secured to the diver's neck by head strap 3100, connecting strap 3125, neck strap 3120, and clasp sections 3130 and 3135 (FIG. 30).

FIG. 32 shows an alternative to securing section 3100 to the diver's neck. A tether strap 3300 is firmly attached to head strap 3100. This attachment is accomplished by sewing strap 3300 to head strap 3100 or other fastener (not shown). Tether 3300 is preferably made of nylon material. It is 20 cm long, and 4 mm in diameter. A C-shaped clip 3305 is fastened to the end of tether 3300. C-shaped clip 3305 is preferably made of plastic, and is 12 mm in diameter and width. FIG. 32 shows tether 3300 as loose and untrained.

Alternatively, tether 3300 can be replaced by a coiled or spring-like tether strap 3400 as shown in FIG. 33. Coil 3400 is also permanently attached to head strap 3100 and to a C-shaped clip 3305.

FIG. 34 shows this embodiment in place on a diver. Mask 100 is held in place by straps 2705, latches 3210, and head strap 3100, as described above. C-shaped clip 3305 is attached to the diver's low-pressure inflator hose 3500. It can also be attached to any other hose crossing the divers shoulder, such as the hose communicating between the air tank and the diver's mouthpiece (not shown). Even if mask 100 is dislodged from the diver's head, it will remain with the diver through the combined actions of straps 2705, latches 3210, posts 3110 (FIG. 33), head strap 3100, tether 3300, and C-shaped clip 3305. If there is no hose 3500 available, then clip 3305 can be replaced with another type of clip or a H&L fastener, in well-known fashion.

As discussed above in connection with connection strap 3125 (FIGS. 30 and 31), clip 3305 can either attach to hose 3500 permanently, or a predetermined pull force on tether 3300 can dislodge it. In the latter case, a diver whose mask becomes entangled in seaweed (for example) can break free if necessary.

FIG. 35 shows an alternative method for securing strap 3525 to head strap 3100 without latch 3210 and post 3110 (FIG. 31). Mask 100 is held in place by strap 3525 that is continuous and extends from one side of mask 100 around the diver's head 3520 to the other side of mask 100. Head strap 3100 is comprised of two thin pieces of neoprene sandwiched together and sewn by stitching 3530 at the perimeter. Ends 3510 of head strap 3100 are not sewn so that strap 3525 can be fed through the head strap 3100 to securely connect strap 3525 to head strap 3100. Conversely strap 3300 can be permanently attached to strap 3525, eliminating head strap 3100 altogether.

Even if mask 100 is dislodged from the diver's head 3520, it will remain with the diver through the combined actions of strap 3525, head strap 3100, tether 3300, and clip 3305.

### Operation—FIGS. 30-35

Mask 100 is attached to posts 3110 on head strap 3100 by straps 2705 and latches 3210. Alternatively, a continuous strap 3525 can be fed through head strap 3100 and can comprise two thin pieces of neoprene sandwiched together with stitching 3530 at the perimeter and open at the ends 3510. Head strap 3100 is held against the back of the wearer's head while mask 100 is either placed on top of the wearer's head or over the eyes. Connecting strap 3125 connects head strap 3100 to neck strap 3120, which is around the wearer's neck so as to prevent loss of mask 100. Alternatively, coil 3400 can connects head strap 3100 to clip 3305, which is in turn secured to hose 3500 or another anchor point to prevent loss of mask 100.

### Eight Embodiment

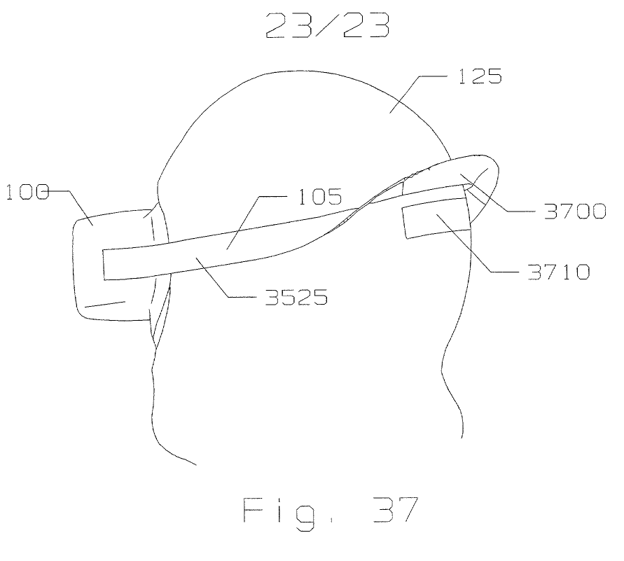

### Alternative Cold-Water Mask Retention Method—FIG. 36

A hood is generally used in cold-water diving. FIG. 36 shows another alternative method for securing strap 3525 to hood 125 to prevent loss of mask 100. A small strap 3600 is sewn to one or both sides of hood 125. Strap 3600 loops around mask strap 3525 and is fed through a plastic loop 3610 attached to one end of strap 3600. Strap 3600 is secured with mating parts of H&L fastener, in well-known fashion. Alternatively, one end of strap 3600 is sewn to hood 125 above strap 3525 and the free end has one half of a hook-and-loop fastener attached. The free end of strap 3600 crosses over mask strap 3525 and mates with the other half of the H&L sewn to the hood. Thus mask strap 3525 is held captive by strap 3600, preventing loss of mask 100 in any position.

### Operation—FIG. 36

The diver places mask 100 on the as shown Strap 3600 is then wrapped around mask strap 3525. The ends of strap 3600 are secured, thus ensuring that mask 100 will not leave the head. This attachment method is very secure and will release only through deliberate action on the diver's part.

### Ninth Embodiment

### Second Alternative Cold-Water Mask Retention Method—FIG. 37

FIG. 37 is the rear side view of a diver wearing a hood and mask in the closed position. One half of a H&L fastener 3700 is attached (sewn or otherwise bonded) to the inside of strap 3525. The other half 3710 of H&L fastener 3700-3710 is attached (sewn or otherwise bonded) to the backside of hood 125 at the position shown Alternatively, one half of a H&L fastener 3700 is attached (sewn or otherwise bonded) to the inside of head strap 3100 (FIG. 35). The other half 3710 of H&L fastener 3700-3710 is attached to the backside of hood 125 at the position shown.

### Operation—FIG. 37

To use this system, the diver merely places mask 100 over the, then stretches strap 3525 over hood 125, joining H&L fastener halves 3700 and 3710. This attachment is not permanent. It will release if the diver pulls as mask 100 becomes entangled in seaweed or other debris.

### Conclusions, Ramifications, and Scope

The present system provides a post-and-latch fastener that is secure, which can be rotated, and which can be detached only through a deliberate action. In a typical application, a pair of posts is affixed to the diver's hood. Two latches attach the straps that connect to the diver's mask. These latches are easily slid over the posts even though the diver is wearing gloves. In all but one embodiment of the present system, the latches can be rotated 360° on the posts without becoming detached. In one embodiment, the latch can be separated from its post only by being rotated approximately 180° and then slidably removed. In all embodiments of the present system, a deliberate action is required to disengage the latch from its post. When not in the water, or on the surface of the water, the diver can move the mask to the top of his or her head without fear of having it fall off. When ready to descend below the surface of the water, the diver can effortlessly move the mask over her or his face, even though wearing gloves. While swimming below the surface of the water, the diver need have no fear of losing the mask due to being kicked in the mask by another diver, bumping into objects, and the like, since these actions are not the deliberate action necessary to separate the latch from the post.

While the above description contains many specificities, these should not be construed as limitations on the scope of the invention, but rather as examples of a presently preferred embodiment and variations. Many other ramifications are possible. For example the fastener system can be used to secure straps on backpacks, guitars, camping gear, dive computers, personal digital assistants, car seats for infants, and the like. Additional ramifications include using the present fastener for attaching cell phones to garments, as a D-ring swivel attachment for use with buoyancy compensating device, a leash connector for attaching a surfboard leash to a wetsuit, a flag connector for use when playing flag football, for mounting or securing lights directly or via a strap to a garment or wetsuit and any other application where two or more objects are tethered.

While the present system employs elements that are well known to those skilled in the art of mechanical engineering, it combines elements in a novel way that produces a new result not heretofore discovered. Accordingly the scope of this invention should be determined, not by the embodiments illustrated, but by the appended claims and their legal equivalents.

I claim:

1. A diver's mask which can easily be positioned in its operative position over a diver's eyes, or moved to an inactive, but ready position on said diver's head above the eyes, comprising:
a. an eye mask having a pair of straps extending from opposite sides thereof, said straps each having a front end attached to said eye mask and a portion distal from said front end which lies adjacent the side of said diver's head,
b. attachment means for attaching said portion distal from said front end to said head of said diver so that eye mask can be moved to said inactive, but ready position without moving said portion distal from said front end away from said side of said diver's head, whereby said mask can be used and easily moved between operative and inactive positions without likelihood of loss.

2. The mask of claim 1 wherein said attachment means comprises a flexible hood covering the head of said diver and a pair of side posts attached to opposite sides of said hood so as to extend out from opposites sides of said diver's head when said hood is worn, said portion distal from said front end of each strap having a latch thereat which can be removably attached to and swivel on a respective one of said posts.

3. The diver's mask of claim 2 wherein each of said side posts comprises a column or shank portion and a ridge or head at the end of said shank distal from said hood, and wherein each of said latches comprises a holding portion having a slot shaped to slide onto said head and a hook for removably snapping over said head when said head is inserted into said slot.

4. The diver's mask of claim 3 wherein said posts each comprise a pair of mating and interlocking flat flange portions with a projection extending perpendicular to said flat member, the projections of said pair of flat flange portions shaped to telescopingly mate with each other, said flat flange portions mounted on opposite sides of said flexible hood, one of said projections extending through a hole in said hood, said flange projections telescopingly mating with each other.

5. The diver's mask of claim 3 wherein said hook comprises a cantilevered finger with said hook at a free end of said finger.

6. The diver's mask of claim 5 wherein said finger extends parallel to said slot.

7. The diver's mask of claim 5 wherein said finger extends perpendicular to said slot.

8. The mask of claim 1 wherein said attachment means comprises a head strap which can be positioned at the back of said diver's head and holding means for securing said mask to said diver in case said mask is inadvertently removed from said diver's head.

9. The mask of claim 8 wherein said holding means comprises a tether strap and a neck collar, said tether strap connecting said head strap to said neck collar.

10. The mask of claim 9 wherein said tether strap is coiled.

11. The mask of claim 8 wherein said holding means comprises a tether strap and means for connecting said tether strap to an article worn by said diver.

12. A diver's mask which can easily be positioned in its operative position over a diver's eyes, or moved to an inactive, but ready position on the diver's head above the eyes, comprising:
a. a flexible hood covering the head of said diver,
b. a pair of side posts attached to opposite sides of said hood so as to extend out from opposites sides of said diver's head when said hood is worn,
c. an eye mask having a pair of straps extending from opposite sides thereof, the end of each strap distal from said eye mask having a latch thereat which can be removably attached to and swivel on a respective one of said posts, whereby said mask can be used and easily moved between operative and inactive positions without likelihood of loss.

13. The diver's mask of claim 12 wherein each of said posts comprises a column or shank portion and a ridge or head at the end of said shank distal from said hood, and wherein each of said latches comprises a holding portion having a slot shaped to slide onto said head and a hook for removably snapping over said head when said head is inserted into said slot.

14. The diver's mask of claim 13 wherein said hook comprises a cantilevered finger with said hook at a free end of said finger.

15. The diver's mask of claim 14 wherein said finger extends parallel to said slot.

16. The diver's mask of claim 14 wherein said finger extends perpendicular to said slot.

17. A diver's mask which can easily be positioned in its operative position over a diver's eyes, or moved to an inactive, but ready position on the diver's head above the eyes, comprising:
a. a flexible hood covering the head of said diver,
b. a pair of side posts attached to opposite sides of said hood so as to extend out from opposites sides of said diver's head when said hood is worn, each of said posts comprising a column or shank portion and a ridge or head at the end of said shank distal from said hood,
c. an eye mask having a pair of straps extending from opposite sides thereof, the end of each strap distal from said eye mask having a Latch thereat which can be removably attached to and swivel on a respective one of said posts, each of said latches comprising a holding portion having a slot shaped to slide onto said head and a hook for removably snapping over said head when said head is inserted into said slot,
whereby said mask can be used and easily moved between operative and inactive positions without likelihood of loss.

18. The diver's mask of claim 17 wherein said hook comprises a cantilevered finger with said hook at a free end of said finger.

19. The diver's mask of claim 18 wherein said finger extends parallel to said slot.

20. The diver's mask of claim 18 wherein said finger extends perpendicular to said slot.

21. A latch which can be removably attached to and swivel on a post, comprising:
a. an inner post with a column or shank and a flat flange at one end,
b. an outer post with a column or shank and a flat flange at one end and a ridge or head distal from said flat flange,
c. a pin with a column or shank and a flange at one end,
d. a latch member with a slot, a finger, and a hook,

22. The latch of claim 21 wherein said inner post has a hollow column that is cut to allow for flexing of said column, said column also having a plurality of recesses along said column, said flat flange having a plurality of projections extending perpendicular to said flat flange member.

23. The latch of claim 21 wherein said outer post has a hollow column and a plurality of ridges along said column, said flat flange having a plurality of projections extending perpendicular to said flat flange.

24. The latch of claim 21 wherein said pin has a solid column with a point on the end distal from said flange, said flange having a recess therein.

25. The latch of claim 21 wherein said post comprises a pair of mating and interlocking flat flange portions with a projection extending perpendicular to said flat member, said projections of said pair of flat flange portions shaped to telescopingly mate with each other.

26. The latch of claim 21 wherein said latch member has a holding portion having a slot shaped to slide onto said head and said hook for removably snapping over said head when said head is inserted into said slot.

27. The latch of claim 26 wherein said hook comprises a cantilevered finger with said hook at a free end of said finger.

28. The latch of claim 26 wherein said finger extends parallel to said slot.

29. The latch of claim 26 wherein said finger extends perpendicular to said slot.
